(19) United States
(12) Patent Application Publication
(10) Pub. No.: US 2024/0071076 A1
(43) Pub. Date: Feb. 29, 2024

(54) METHODS, SYSTEMS AND MEDIUMS FOR SURGICAL AUTOMATION

(71) Applicant: SHANGHAI UNITED IMAGING INTELLIGENCE CO., LTD., Shanghai (CN)

(72) Inventors: Terrence CHEN, Cambridge, MA (US); Ziyan WU, Cambridge, MA (US); Shanhui SUN, Cambridge, MA (US); Arun INNANJE, Cambridge, MA (US); Meng ZHENG, Cambridge, MA (US); Benjamin PLANCHE, Cambridge, MA (US); Abhishek SHARMA, Cambridge, MA (US)

(73) Assignee: SHANGHAI UNITED IMAGING INTELLIGENCE CO., LTD., Shanghai (CN)

(21) Appl. No.: 17/823,950

(22) Filed: Aug. 31, 2022

(51) Int. Cl. G06V 20/40; G06V 20/52

(52) U.S. Cl. CPC G06V 20/41; G06V 20/44; G06V 20/52; A61B 34/30

(57) ABSTRACT

The present disclosure provides a method for surgical automation. The method may include: obtaining video data generated in a surgical process using a camera; identifying a surgical stage in the surgical process based on the video data; and triggering an activation of a surgical equipment used for a surgical operation and/or providing a guidance of the surgical operation based on the surgical stage.

### TECHNICAL FIELD

The present disclosure relates to the field of medical treatment, in particular, to a method, a system and medium for surgical automation.

### BACKGROUND

In the current medical treatment system, surgeons generally learn surgical operations from books, videos or senior surgeons' surgical operations during training or before performing a surgical operation independently. During the surgical process, a surgeon needs to select different surgical equipment based on the patient's condition, and complete the surgical process by referring to different surgical images. The selection of surgical equipment and surgical images needs to be manually implemented by the surgeon, and the surgeon may not get a real-time guidance during the surgical process. Therefore, it is desirable to provide a method for surgical automation of the surgical process and provide a real-time guidance during the surgical process.

### SUMMARY

One aspect of the present disclosure may provide a method for surgical automation. The method may include: obtaining video data generated in a surgical process using a camera; identifying a surgical stage in the surgical process based on the video data; and triggering an activation of a surgical equipment used for a surgical operation and/or providing a guidance of the surgical operation based on the surgical stage.

Another aspect of the present disclosure may provide a system for surgical automation. The system may include a first acquisition module configured to obtain video data generated in a surgical process using a camera; an identification module configured to identify a surgical stage in the surgical process based on the video data; and a triggering and displaying module configured to trigger an activation of a surgical equipment used for a surgical operation and/or provide a guidance of the surgical operation based on the surgical stage.

Another aspect of the present disclosure may provide a non-transitory computer readable medium. The non-transitory computer readable medium may store at least one set of instructions, wherein when executed by at least one processor of a computing device, the at least one set of instructions directs the at least one processor to perform the method for surgical automation.

### BRIEF DESCRIPTION OF THE DRAWINGS

The present disclosure is further illustrated in terms of exemplary embodiments, and these exemplary embodiments are described in detail with reference to the drawings. These embodiments are not limited. In these embodiments, the same numeral indicates the same structure, wherein:

FIG. 1 is a schematic diagram illustrating an exemplary application scenario of a system for surgical automation according to some embodiments of the present disclosure;

FIG. 2 is a block diagram illustrating an exemplary system for surgical automation according to some embodiments of the present disclosure;

FIG. 3 is a flowchart illustrating an exemplary process of a method for surgical automation according to some embodiments of the present disclosure;

FIG. 4 is a schematic diagram illustrating an exemplary model of identifying a surgical stage in a surgical process according to some embodiments of the present disclosure;

FIG. 5 is a flowchart illustrating an exemplary process of determining target guidance information according to some embodiments of the present disclosure;

FIG. 6 is another flowchart illustrating an exemplary process of determining target guidance information according to some embodiments of the present disclosure;

FIG. 7 is a flowchart illustrating an exemplary process of providing prompt information according to some embodiments of the present disclosure; and

FIG. 8 is a schematic diagram illustrating an exemplary process of determining an execution level by weighting according to some embodiments of the present disclosure.

### DETAILED DESCRIPTION

In order to illustrate the technical solutions related to the embodiments of the present disclosure, brief introduction of the drawings referred to in the description of the embodiments is provided below. Obviously, drawings described below are only some examples or embodiments of the present disclosure. Those having ordinary skills in the art, without further creative efforts, may apply the present disclosure to other similar scenarios according to these drawings. Unless stated otherwise or obvious from the context, the same reference numeral in the drawings refers to the same structure and operation.

It will be understood that the terms “system,” “device,” “unit,” and/or “module” used herein are one method to distinguish different components, elements, parts, sections, or assemblies of different levels in ascending order. However, the terms may be displaced by other expressions if they may achieve the same purpose.

As shown in the present disclosure and claims, unless the context clearly indicates exceptions, the words “a,” “an,” “one,” and/or “the” do not specifically refer to the singular, but may also include the plural. The terms “including” and “comprising” only suggest that the steps and elements that have been clearly identified are included, and these steps and elements do not constitute an exclusive list, and the method or device may also include other steps or elements.

The flowcharts used in the present disclosure may illustrate operations executed by the system according to embodiments in the present disclosure. It should be understood that a previous operation or a subsequent operation of the flowcharts may not be accurately implemented in order. Conversely, various operations may be performed in inverted order, or simultaneously. Moreover, other operations may be added to the flowcharts, and one or more operations may be removed from the flowcharts.

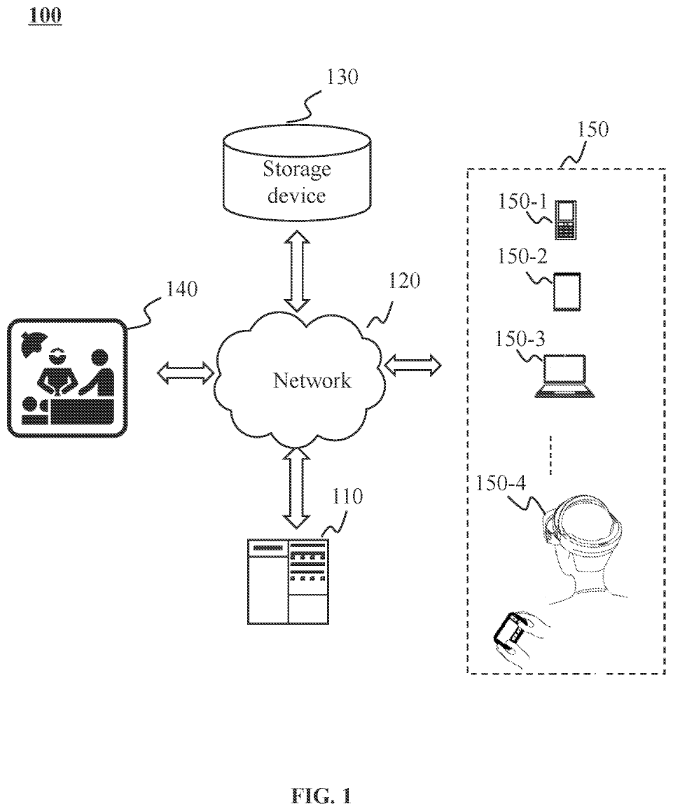

FIG. 1 is a schematic diagram illustrating an exemplary application scenario of a system for surgical automation according to some embodiments of the present disclosure. The surgical automation may mean that a surgical equipment for surgical operation is operated automatically during a surgical process. For example, the surgical equipment may automatically move to a standby position and perform a surgical operation spontaneously. Meanwhile, the surgical automation may also include automatically determining a guidance of the surgical operation. As shown in FIG. 1, in some embodiments, an application scenario 100 of the system for surgical automation may include a processing device 110, a network 120, a storage device 130, an operating room 140, and a user terminal 150.

In some embodiments, one or more components of the application scenario 100 may be connected and/or communicate with each other via the network 120, such as a wireless connection, a wired connection, or a combination thereof. As shown in FIG. 1, the processing device 110 may be connected with the storage device 130 via the network 120. For another example, the processing device 110 may be connected with the user terminal 150 via the network 120.

The processing device 110 may be configured to process information and/or data related to the application scenario 100, for example, medical image information, parameter information of the surgical equipment, patient physiological data, or the like. The processing device 110 may process data, information, and/or a processing result obtained from other devices or components, and execute instructions based on these data, information, and/or the processing result to perform one or more functions described in the present disclosure. In some embodiments, the processing device 110 may acquire information such as patient case data stored in the storage device 130 and/or input by the user terminal 150. In some embodiments, the processing device 110 may determine a surgical equipment based on the acquired information and/or data.

The network 120 may connect with various components of the application scenario 100 and/or connect the application scenario 100 with an external resource. The network 120 may allow a communication between various components, or between the application scenario 100 and the external resource, to facilitate the exchange of data and/or information. For example, the network 120 may transmit data in the storage device 130 to the processing device 110. In some embodiments, the network 120 may include, but be not limited to, a wired network or a wireless network, or the like.

The storage device 130 may be configured to store data and/or instructions. In some embodiments, the storage device 130 may store data and/or instructions used by the processing device 110 to execute or complete methods described in the present disclosure. In some embodiments, the storage device 130 may be connected with the network 120 to communicate with one or more components of the application scenario 100.

The operating room 140 may refer to a room where a surgical operation is performed. In some embodiments, the operating room may include a camera, an operating table, a medical imaging equipment, a surgical equipment, or the like. The medical imaging device may include an angiographic system, an ultrasound device, or the like. The surgical equipment may include a surgical robot, a movable C-arm, or the like.

In some embodiments, the camera in the operating room 140 may acquire video stream data of the surgical process and transmit the video stream data to the processing device 110 through the network 120. In some embodiments, the surgical robot may automatically move from a standby position to a working position of a surgical stage in response to an instruction transmitted by the processing device 110. The medical imaging device may change position or angle to acquire a desired image.

The user terminal 150 may be configured to input information and/or data related to the application scenario 100, for example, the patient's case information, the parameter of the surgical equipment, or the like. The user terminal 150 may also be configured to receive information and/or data related to the application scenario 100, for example, the guidance of the surgical operation, operation prompt information, or the like. In some embodiments, the user terminal 150 may present the received information and/or data, for example, the received information and/or data may be displayed on a screen of the terminal display by displaying a video, a picture, or the like.

In some embodiments, the user terminal 150 may include at least one of the mobile device 150-1, the tablet computer 150-2, the notebook computer 150-3, or any combination thereof. In some embodiments, the user terminal 150 may also include an extended reality (XR) device 150-4, such as a pair of virtual reality (VR)/augmented reality (AR) glasses. The XR device may display an image, a model, a video, or other information, and may be used for remote surgical guidance.

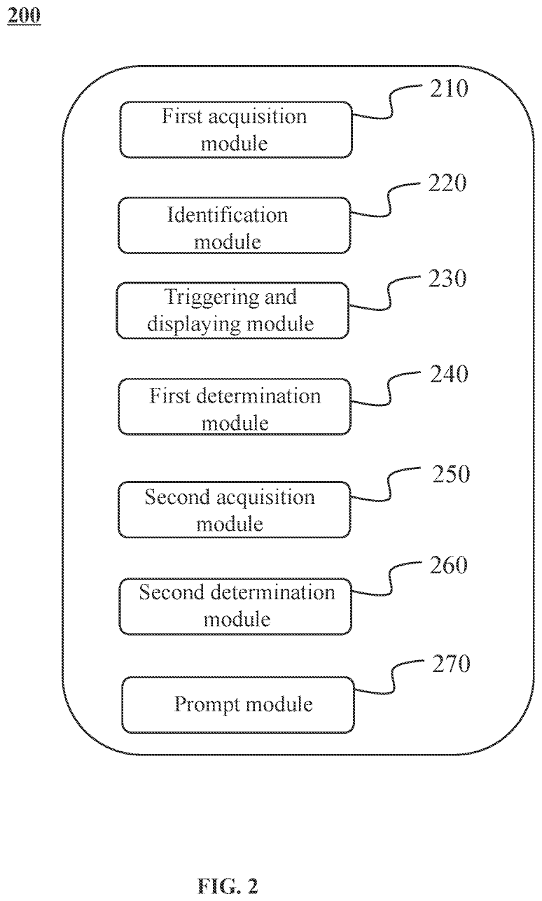

FIG. 2 is a block diagram illustrating an exemplary system for surgical automation according to some embodiments of the present disclosure. As shown in FIG. 2, a system 200 for surgical automation may include a first acquisition module 210, an identification module 220, and a triggering and displaying module 230.

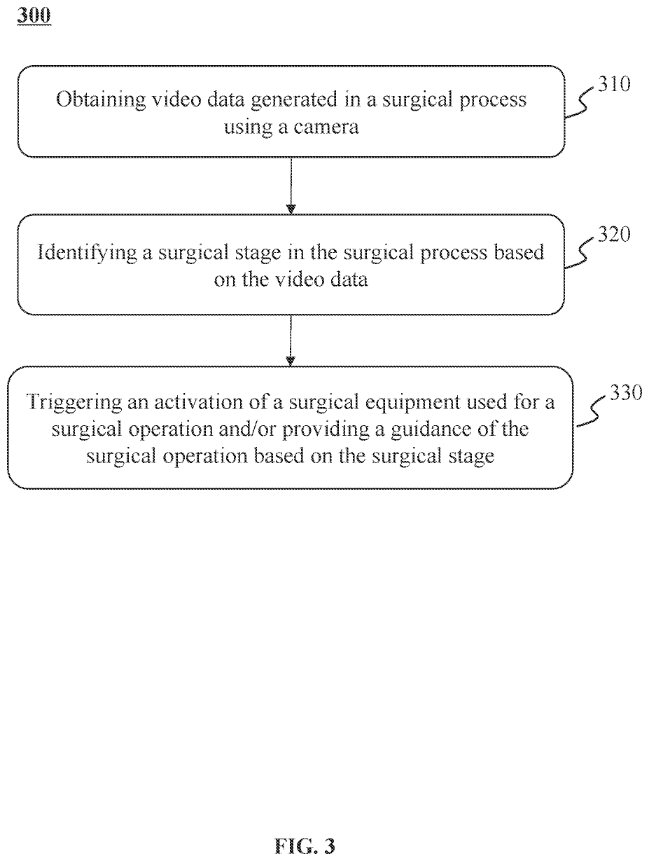

The first acquisition module 210 may be configured to obtain video data generated in a surgical process using a camera. More descriptions of the video data generated in the surgical process using the camera may be found elsewhere in the present disclosure, for example, FIG. 3 and the descriptions thereof.

The identification module 220 may be configured to identify a surgical stage in the surgical process based on the video data. In some embodiments, the surgical stage may include normal surgical stage and/or an abnormal surgical accident.

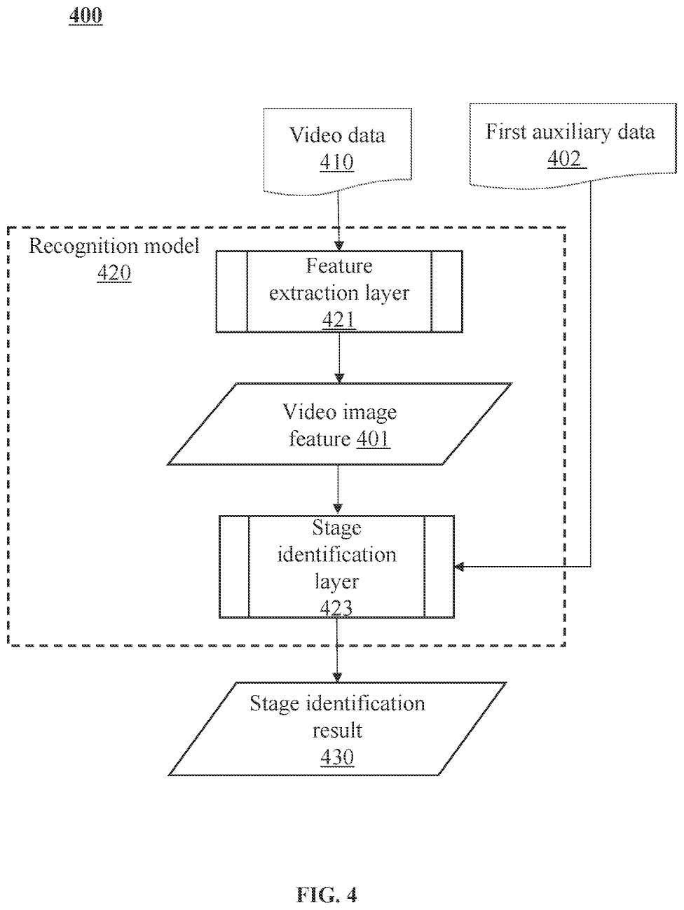

In some embodiments, the identification module 220 may input the video data into a recognition model, and determine the surgical stage based on an output of the recognition model. The recognition model may be a machine learning model. More descriptions of identifying the surgical stage may be found elsewhere in the present disclosure, for example, FIG. 3, FIG. 4 and the descriptions thereof.

The triggering and displaying module 230 may be configured to trigger an activation of a surgical equipment used for a surgical operation and/or provide a guidance of the surgical operation based on the surgical stage.

In some embodiments, the triggering and displaying module 230 may also be configured to determine the surgical equipment used for the surgical operation and/or the guidance of the surgical operation based on target guidance information.

In some embodiments, the triggering and displaying module 230 may also be configured to transmit an activation instruction to the surgical equipment used for the surgical operation, and the activation instruction may include one or more parameters. More information of triggering an activation of a surgical equipment used for a surgical operation and/or providing a guidance of the surgical operation may be found elsewhere in the present disclosure, for example, FIG. 3 and the description thereof.

In some embodiments, the system 200 for surgical automation may further include a first determination module 240. The first determination module 240 may be configured to determine target guidance information based on the surgical stage.

In some embodiments, the first determination module 240 may further configured to determine the target guidance information from a plurality of pieces of standard guidance information based on a matching relationship between the surgical stage and reference information corresponding to the plurality of pieces of standard guidance information, and the reference information may include a reference surgical stage.

In some embodiments, the first determination module 240 may also be configured to obtain second auxiliary data, the second auxiliary data may include case data of a subject, and/or physiological monitoring data related to an acquisition time of the video data. The first determination module 240 may also be configured to determine the target guidance information based on the surgical stage, and a matching relationship between the second auxiliary data and the reference information.

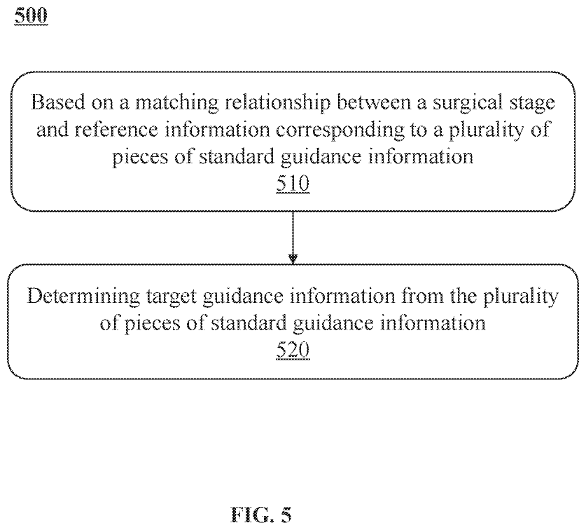

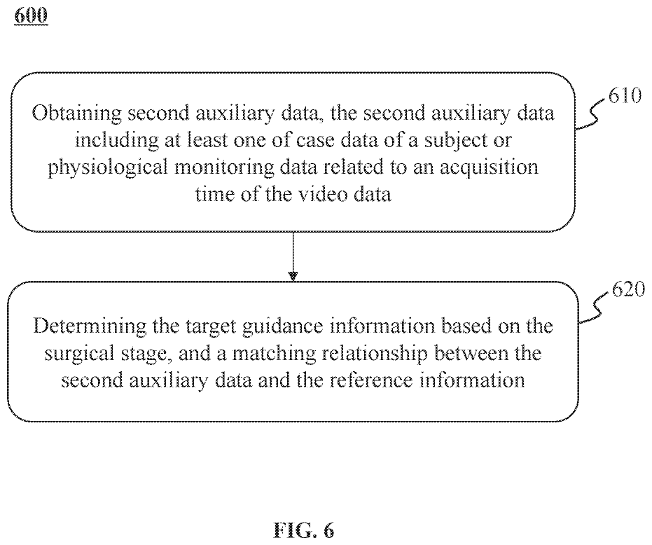

More descriptions of how to determine the target guidance information may be found elsewhere in the present disclosure, for example, FIG. 5, FIG. 6 and the descriptions thereof.

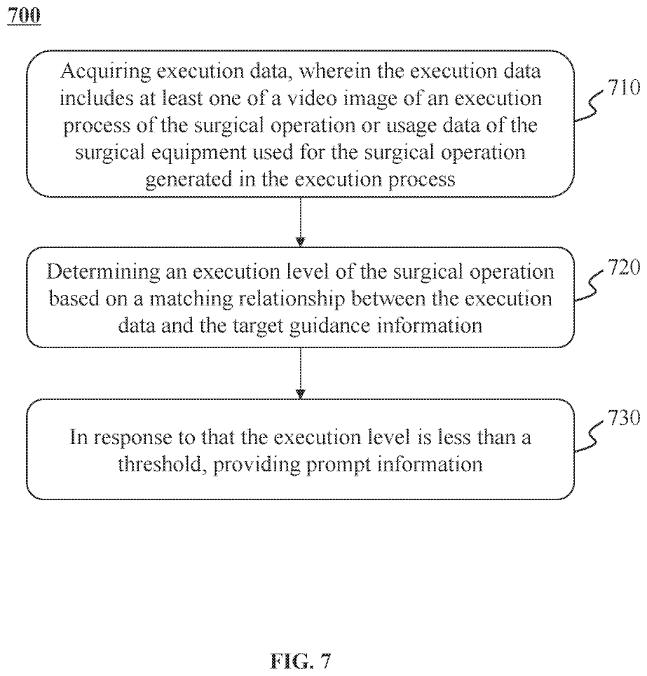

In some embodiments, the system 200 for surgical automation may further include a second acquisition module 250. The second acquisition module 250 may be configured to acquire execution data, and the execution data may include at least one of a video image of an execution process of the surgical operation or usage data of the surgical equipment used for the surgical operation generated in the execution process. More descriptions of acquiring execution data may be found elsewhere in the present disclosure, for example, FIG. 7 and the descriptions thereof.

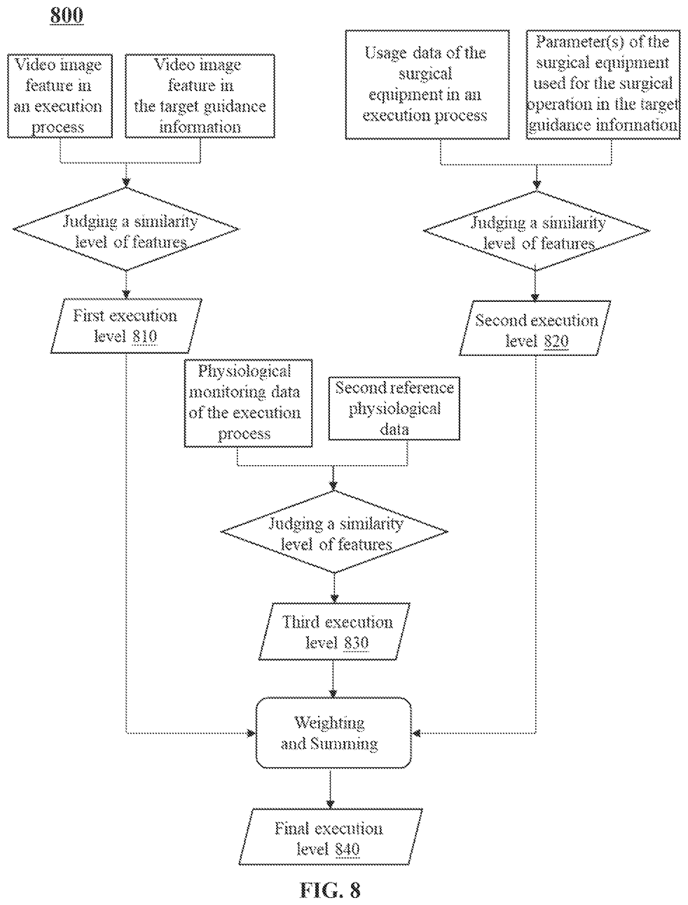

In some embodiments, the system 200 for surgical automation may further include a second determination module 260. The second determination module 260 may be configured to determine an execution level of the surgical operation based on a matching relationship between the execution data and the target guidance information. More descriptions of determining the execution level may be found elsewhere in the present disclosure, for example, FIG. 7, FIG. 8 and the descriptions thereof.

In some embodiments, the system 200 for surgical automation may further include a prompt module 270. The prompt module 270 may be configured to, in response to that the execution level is less than a threshold, provide prompt information. More descriptions of providing the prompt information may be found elsewhere in the present disclosure, for example, FIG. 7 and the descriptions thereof.

It should be noted that the above description of the system for surgical automation and modules is merely for convenience of description, and may not limit the scope of the present disclosure. It should be understood that, for those skilled in the art, after understanding the principle of the system, it is possible to arbitrarily combine various modules without departing from this principle, or form a sub-system to connect with other modules. In some embodiments, the first determination module and the second determination module disclosed in FIG. 2 may be different modules in a system, or may be a module to implement the functions of two or more modules described above. For example, each module may share one memory module, or each module may have its own memory module. Such modifications are within the scope of the present disclosure.

FIG. 3 is a flowchart illustrating an exemplary process of a method for surgical automation according to some embodiments of the present disclosure. As shown in FIG. 3, process 300 may include one or more of the following operations.

In 310, video data generated in a surgical process may be obtained using a camera. In some embodiments, operation 310 may be performed by the first acquisition module 210.

The camera may refer to a video acquisition device or an image acquisition device arranged in an operating room. The camera may be an RGB camera, an RGBD camera, a panoramic camera, a thermal sensor, or other optical sensor enable to acquire a series of videos or images. In some embodiments, the camera may be arranged at any position where the surgical process may be observed, such as a corner of a ceiling. In some embodiments, a photographing angle of the camera may be changed as required. For example, a participant of the surgical process may adjust the photographing angle and one or more parameters of the camera based on a required image. For another example, the camera may automatically track a motion of a doctor's hand, and conduct directional photographing.

In some embodiments, the camera may also be a wearable device that may display an instruction for a surgical operation, such as a virtual reality (VR) device or a pair of augmented reality (AR) glasses.

Using the wearable device to obtain video images may provide more ways to obtain video data generated in the surgical process. Meanwhile, since a medical staff who performs surgical operations wearing the wearable device is close to the operating table, a photographing operation may be performed at a clearer angle and a closer distance. And images may be photographed from an angle of view of the medical staff, thus the images seen by the medical staff may be obtained, and further image analysis may be performed.

The video data may refer to video data recording the surgical process. The video data may include information such as a surgical operation of a doctor, a use of a medical equipment, or a state of a patient's surgical site. In some embodiments, the video data may be video stream data obtained by a camera.

In some embodiments, the first acquisition module 210 may receive the video data generated in the surgical process using the camera. For example, the camera may transmit the obtained real-time video data to the first acquisition module, and may also transmit the obtained video data within a time period to the first acquisition module. For another example, a doctor may independently select the video data obtained by the VR device/AR glasses and upload the video data to the first acquisition module.

In 320, a surgical stage in the surgical process may be identified based on the video data. In some embodiments, operation 320 may be performed by the identification module 220.

The surgical stage may refer to a surgical progress of the surgical process. In some embodiments, the surgical process may be divided into different surgical stages based on different classification standards. For example, based on the whole surgical process, the surgical stage may be divided into preoperative preparation, intraoperative operation, postoperative suture, or other stages. For another example, the surgical stage may be divided based on a surgical type. For a cholecystectomy, the surgical stage may be divided into placing tools, cutting off a gallbladder, gallbladder packing, coagulation cleaning, pulling out the gallbladder, or other stages.

In some embodiments, the surgical stage may also be divided into a normal surgical stage and/or an abnormal surgical accident based on whether an abnormal accident occurs.

The normal surgical stage may refer to a surgical stage conforming to a normal surgical process. A complete set of surgical process may be composed of a plurality of normal surgical stages, which may facilitate a successful completion of a surgery.

The abnormal surgical accident may refer to a surgical stage that does not conform to the normal surgical process. For example, the abnormal surgical accident may include a sudden condition, such as massive bleeding during the surgical process. For another example, the abnormal surgical accident may include an equipment failure, a surgical error caused by a doctor, or the like.

In some embodiments, the identification module 220 may identify the surgical stage in a variety of ways. In some embodiments, the surgical stage may be determined based on a manual judgment. For example, the doctor may input a judgment result of the surgical stage to the identification module through the user terminal. For another example, the identification module 220 may determine the surgical stage in combination with an overall estimated duration and a currently performed duration of the surgical process. In some embodiments, the identification model may be configured to determine the surgical stage. For example, information such as a video image and voice may be input into a recognition model to determine the surgical stage. More descriptions of determining the surgical stage based on the recognition model may be found elsewhere in the present disclosure, for example, FIG. 4 and the descriptions thereof.

In 330, an activation of a surgical equipment used for a surgical operation may be triggered and/or a guidance of the surgical operation may be provided based on the surgical stage. In some embodiments, operation 330 may be performed by the triggering and displaying module 230.

The surgical equipment may refer to a surgical equipment that needs to be used in a surgical stage, such as a surgical robot used for a surgical operation, an imaging device used for surgical site observation, or the like.

The guidance of the surgical operation may refer to guidance information related to the surgical operation. The guidance of the surgical operation may include information such as a surgical method, a surgical operation angle, or a surgical operation strength. For example, the guidance of the surgical operation in thoracotomy may include a direction and a size of an incision, an operation method of peeling a connective tissue, or the like. The guidance of the surgical operation may be in many forms, such as a video, an audio, an image, or a text.

In some embodiments, the first determination module 240 may determine the surgical equipment for the surgical operation and the guidance of the surgical operation based on a variety of methods. For example, the first determination module 240 may determine the surgical equipment for the surgical operation and the guidance of the surgical operation based on a remote guidance of an expert during a live surgical process. For example, when a live cholecystectomy is performed, an expert who watch the live cholecystectomy remotely may provide a guidance on a use of a resection tool and/or a resection operation in real time.

In some embodiments, the first determination module 240 may determine the surgical equipment for the surgical operation and the guidance of the surgical operation based on the target guidance information determined based on the surgical stage.

The target guidance information may refer to information that guides a surgical stage. The target guidance information may include video, picture, voice, text, or other forms of information. The target guidance information may be determined in combination with the information of the surgical stage. For example, the surgical stage is an abnormal operation accident with massive bleeding of the patient, and a corresponding target guidance information may be video and audio information related to coagulation treatment method(s), parameter(s) of a blood transfusion equipment, or the like. For another example, when the surgical stage is postoperative suture, a corresponding target guidance information may be a graphical description of a suture method, a summary of key points of the suture, or the like.

In some embodiments, the first determination module 240 may determine the target guidance information in a variety of ways. For example, the first determination module 240 may determine the target guidance information based on the surgical stage and the surgical operation. For example, the first determination module 240 may determine the target guidance information as a thoracotomy position, a direction and/or a size of an incision based on the surgical stage being a thoracotomy stage and the surgical operation being a doctor picking up a high-frequency electric knife. For another example, the first determination module 240 may determine that the target guidance information includes setting parameters (such as a power and an argon flow of a high-frequency electric knife equipment) based on the surgical stage being the thoracotomy stage and the surgical operation being the doctor picking up the high-frequency electric knife. For another example, the first determination module 240 may determine the target guidance information based on physiological data of the patient. For example, the first determination module 240 may determine the target guidance information as a blood transfusion operation based on a rapid loss of the patient's red blood cells. For another example, the first determination module 240 may determine that the target guidance information is a tidal volume, a flow rate, or other parameters of a ventilator based on a rapid decrease of the patient's blood oxygen content. More description of determining the target guidance information may be found elsewhere in the present disclosure, for example, FIG. 5, FIG. 6 and the descriptions thereof.

In some embodiments, triggering the activation of the surgical equipment for the surgical operation may include triggering the surgical equipment for surgical operation to enter a standby state and/or move to a standby position. For example, the triggering and displaying module 230 may transmit a standby instruction to the surgical equipment to move the surgical equipment to the front of the operating table.

In some embodiments, triggering the activation of the surgical equipment used for the surgical operation may include transmitting an activation instruction to the surgical equipment used for the surgical operation.

The activation instruction may refer to an instruction that triggers the activation of the surgical equipment used for surgical operation. In some embodiments, the activation instruction may include information such as an activation time, a position when the surgical equipment is used, or the like. For example, the activation instruction may indicate switching on a surgical robot at nine o'clock, and moving the surgical robot to a chest position of the patient when the surgical robot is active.

In some embodiments, the activation instruction may further include one or more parameters. The one or more parameters may refer to one or more parameters of the surgical equipment used for the surgical operation. In some embodiments, the one or more parameters of the surgical equipment used for the surgical operation may be set in advance based on the target guidance information. For example, for a chest angiography, a contrast medium flow rate of an angiography device may be set in advance as 6-8 ml/s based on guidance information of the chest angiography. In some embodiments, the one or more parameters of the surgical equipment used for the surgical operation may be adjusted based on a surgical condition. For example, for the above chest angiography, when an angiographic vessel changes to a pulmonary aorta, the contrast medium flow rate may be adjusted to 18-20 ml/s.

In some embodiments, the triggering and displaying module 240 may provide the guidance of the surgical operation in a variety of ways. For example, the guidance of surgical operation may be provided on a display screen or a projector in the operating room. For another example, the guidance of the surgical operation may be provided on the VR device/AR glasses worn by the doctor. In some embodiments, the guidance showing the surgical operation may support a personalized operation of the user. For example, displayed image information may be enlarged, reduced or rotated, a video progress bar may be dragged for repeated viewing, and audio information may be converted into text.

In some embodiments, providing the guidance of the surgical operation may further include providing a plurality of prompt information, and the prompt information may be determined based on the surgical stage.

The prompt information may be information related to prompting whether the surgical stage is normal or not, and/or information related to the surgical operation corresponding to the surgical stage. In some embodiments, the prompt information may prompt the doctor whether the surgical operation is abnormal. For example, when a doctor's surgical operation deviates from the normal surgical process, the prompt information may be provided. In some embodiments, the prompt information may prompt the patient whether a physiological state is abnormal. For example, when a certain physiological data of the patient changes abnormally or exceeds a safety range, a prompt information may be provided. In some embodiments, the prompt information may prompt the doctor of the surgical operation corresponding to the surgical stage. For example, when entering a certain surgical stage, the prompt information may be provided automatically to show a required surgical operation corresponding to the surgical stage or explain some precautions in the surgical operation corresponding to the surgical stage. In some embodiments, the prompt information may be flexible prompt information to avoid affecting a normal work of the doctor. More descriptions of the prompt information may be found elsewhere in the present disclosure, for example, FIG. 7 and the description thereof.

Based on the surgical stage, the prompt information may help the doctor adjust an improper operation in the surgical process and find changes of the patient's physical condition in time, to ensure a smooth surgical process.

The method for surgical automation shown in some embodiments of the present disclosure may determine a surgical stage and provide guidance of a surgical operation to help a doctor perform a surgical process smoothly. At the same time, based on automatically triggering a standby and use of the surgical equipment based on the surgical stage, time for a medical staff to operate the surgical equipment may be saved, and the surgical efficiency may be improved.

FIG. 4 is a schematic diagram illustrating an exemplary model of identifying a surgical stage in a surgical process according to some embodiments of the present disclosure.

In some embodiments, identifying the surgical stage in the surgical process may include inputting video data 410 into a recognition model 420, and determining the surgical stage based on an output of the recognition model. The recognition model may be a machine learning model.

The recognition model may be configured to identify the surgical stage. In some embodiments, the recognition model 420 may include a feature extraction layer 421 and a stage identification layer 423. The feature extraction layer may be of a convolutional neural network (CNN) structure, and the stage identification layer may be of a recurrent neural network (RNN) structure.

The feature extraction layer 421 may be configured to extract a video image feature 401. An input of the feature extraction layer may include video data 410, and an output of the feature extraction layer may include the video image feature 401. The video image feature may be feature information related to identifying the surgical stage and extracted from the video data. The video image feature may include information such as the doctor's hand operation, a state of the patient's surgical site, or a type of the surgical equipment. When the input video data is video image data at a certain time, the output video image feature may be a video image feature at that time. When the input video data is video data for a period of time, the output video image feature may be a video image feature sequence corresponding to an acquisition time of the input video data.

The video data 410 may refer to video data recording the surgical process. More descriptions of the video data may be found elsewhere in the present disclosure, for example, FIG. 3 and the descriptions thereof.

The video image feature may be represented by a feature vector. For example, a vector \vec{m}=(a, b, c) may be constructed, where a represents information of the doctor's hand operation, b represents information of a state of the patient's surgical site, and c represents information of a type of the surgical equipment.

The stage identification layer 423 may be configured to determine the surgical stage. The input of the stage identification layer may include a video image feature 401, which may be a sequence of video image features determined based on the acquisition time of the video image. For example, the video image feature sequence may be expressed as {mₜ₁, mₜ₂ . . . }, where each element represents a video image feature corresponding to an acquisition time. The output of the stage identification layer may include a stage identification result 430.

The stage identification result 430 may refer to an output of the recognition model, that is, the surgical stage determined based on the recognition model, such as a “preoperative preparation” stage, a “surgical pretreatment” stage, a “postoperative suture” stage, or the like. More descriptions of the surgical stage may be found elsewhere in the present disclosure, for example, FIG. 3 and the descriptions thereof.

In some embodiments, different types of the surgical operation may have different surgical stages. Based on different types of the surgical operation, different recognition models may be configured to obtain different surgical stages corresponding to different types of the surgical operation. For example, for a thoracotomy, a corresponding thoracotomy stage may be obtained using a thoracotomy recognition model. For a cholecystectomy, a corresponding cholecystectomy stage may be obtained using a cholecystectomy recognition model.

In some embodiments, the input of the recognition model may further include first auxiliary data 402 related to the acquisition time of the video data.

The acquisition time of the video data may refer to an acquisition time of the video data input to the recognition model. For example, if video data input to the recognition model is obtained at 09:00, then 09:00 is the acquisition time of the video data. In some embodiments, the first acquisition module 210 may save the acquisition time of the video data while extracting the video data. For example, the first acquisition module 210 may obtain the video data at 09:00, and store the information that the acquisition time is 09:00.

The first auxiliary data may refer to data used to assist in determining the surgical stage. The first auxiliary data used to determine the surgical stage may be data whose acquisition time corresponds to the video data acquisition time. For example, if the acquisition time of video data is 09:00, the first auxiliary data is auxiliary data obtained at 09:00. In some embodiments, the first auxiliary data may include audio data, physiological monitoring data, a type of a surgical equipment, usage data of the surgical equipment, or the like, or a combination thereof.

The audio data may refer to recognition data of communication audio between medical staffs during the surgical process. The audio data may include “the surgical operation may be started”, “the patient is bleeding, ready for blood transfusion”, “ready for suturing”, or the like. In some embodiments, the audio data and video data may be input into the recognition model to obtain the stage recognition result. For example, the audio data of “ready for suturing” and the video data when the doctor picks up a suturing bag may be input into the recognition model, and the stage recognition result may be a surgical suturing stage.

The physiological monitoring data may refer to patient's physiological data received by a monitoring device during the surgical process, such as a patient's heart rate, respiration, a blood pressure, or other data. In some embodiments, the physiological monitoring data and video data may be input into the recognition model to obtain the stage recognition result. For example, the physiological monitoring data of a patient's red blood cell concentration and the video data of the doctor operates a blood transfusion equipment may be jointly input into the recognition model, and the stage recognition result may be a blood transfusion stage.

The type of the surgical equipment and the usage data of the surgical equipment may refer to a type and usage data of the surgical equipment used in the surgical process. For example, the type of the surgical equipment may be a surgical robot, a movable C-arm, or the like. The usage data may include a working angle, coordinates, and a usage parameter of the surgical equipment. In some embodiments, the type of the surgical equipment, the usage data of the surgical equipment, and video data may be input into the recognition model to obtain the stage recognition result. For example, a type being a high-frequency electric knife, a power of the high-frequency electric knife, and the video data of the doctor cutting chest skin with the high-frequency electric knife may be jointly input into the recognition model, and the stage recognition result may be an opening chest stage.

In some embodiments, the first auxiliary data may be obtained based on a variety of ways. For example, the audio data may be obtained through third-party voice recognition software, the physiological monitoring data can be obtained through the corresponding physiological monitoring equipment, and the type of surgical equipment and the use data of surgical equipment can be obtained by identifying the pictures taken by the camera and the relevant information of the surgical equipment.

In some embodiments, the audio data may also be obtained based on an audio recognition model.

The audio recognition model may be configured to determine an audio recognition result, that is, to obtain the audio data. The audio recognition model may be a trained machine learning model. The audio recognition model may include a cyclic neural network model, a deep neural network model, or the like, or any combination thereof.

The input of the audio recognition model may include communication audio, and the output of the audio recognition model may include the audio recognition result. In some embodiments, the audio recognition result may be represented by a text. That is, the audio recognition model may convert the communication audio into a corresponding text.

In some embodiments, training data of the audio recognition model may be a plurality of labeled second training samples. The plurality of second training samples may be historical communication audio, and the communication audio may come from a camera or other recording devices. A training label of a second training sample of the audio recognition model may be an audio recognition result, and the training label may be obtained by manual annotation. In some embodiments, a loss function may be constructed based on a training label and an initial audio recognition result. A parameter of the audio recognition model may be iteratively updated by gradient descent or other methods based on the loss function. When a predetermined condition is met, a training of the audio recognition model may be completed, and the trained audio recognition model may be obtained. The predetermined condition may be convergence of the loss function, a count of iterations reaching a threshold, or the like.

In some embodiments, the first auxiliary data and the video data may be used as input of the recognition model to identify the surgical stage.

In some embodiments, the recognition model may further include an embedding layer. The audio recognition result may be input into the embedding layer, and an audio feature may be output. The audio feature may include an audio feature sequence corresponding to an acquisition time of the communication audio. The audio feature may be represented by a feature vector. The audio feature vector may represent a relevant feature of communication in the operating room. For example, the audio feature sequence may be expressed as {Vₜ₁, Vₜ₂ . . . }, where each element represents an audio feature vector at a corresponding time. In some embodiments, the audio feature and the video image feature may be input to the stage identification layer to determine the surgical stage.

In some embodiments, the physiological monitoring data, the type of the surgical equipment and the usage data of the surgical equipment may be input into the stage identification layer. The physiological monitoring data, the type of the surgical equipment and the usage data of the surgical equipment may be data sequences corresponding to an acquisition time. For example, a sequence of the physiological monitoring data may be expressed as {Dₜ₁, Dₜ₂ . . . } wherein each element represents physiological monitoring data corresponding to the acquisition time. The stage identification layer may identify the surgical stage based on the video image feature, the audio feature, the physiological monitoring data, the type of the surgical equipment and usage data of the surgical equipment.

By inputting the first auxiliary data such as the audio data into the recognition model, the identification of the surgical stage may be facilitated, thus the accuracy of identifying the surgical stage may be improved, and more accurate guidance information may be provided to help to facilitate the surgical process.

In some embodiments, the recognition model 420 may be obtained based on training.

In some embodiments, the feature extraction layer and the stage identification layer of the recognition model may be separately trained.

In some embodiments, the feature extraction layer may be pre-trained. For example, the feature extraction layer obtained by pre-training may be obtained by training an image recognition model. The image recognition model may include a feature extraction layer and a type recognition layer. The video data or images may be input into the image recognition model to obtain types of objects in the video data or images, for example, a type may include a heart, a hand, or the like.

In some embodiments, the image recognition model may be trained based on a large count of labeled training data. The training data of the image recognition model may be sample video data or the sample image, and a label may be a type of an object in sample video data or a sample image. The training data and the label of the image recognition model may be derived from historical video image data. For example, the sample video data or the sample image may be input to the feature extraction layer to obtain a sample video image feature output by the feature extraction layer. Then, the sample video image feature output by the feature extraction layer may be input to the type recognition layer to obtain sample video data or a type of the object in the sample image output by the type recognition layer. A parameter of an initial feature extraction layer and the initial type recognition layer may be updated through training until an intermediate feature extraction layer and an intermediate type recognition layer meet a predetermined condition, and a trained image recognition model may be obtained. The feature extraction layer of the trained image recognition model may be designated as the pre-trained feature extraction layer. The predetermined condition may be that a loss function is less than a threshold or converges, or a training period reaches a threshold.

The training data and the label of the image recognition model may be easy to obtain. Training the feature extraction layer based on the image recognition model may improve a training speed of the feature extraction layer.

In some embodiments, after obtaining the pre-trained feature extraction layer, the stage identification layer may be trained based on first training sample(s) and label(s) of the first training sample(s). The first training sample(s) may include the sample video data, and the label(s) may include an actual surgical stage. The sample video data may be input to the pre-trained feature extraction layer, and the sample video image features may be output by the pre-trained feature extraction layer to the stage identification layer to be trained (i.e., an initial stage identification layer). A loss function may be constructed based on a prediction result (i.e., the stage recognition result) and the label output by the stage identification layer to be trained. A parameter of the stage identification layer to be trained may be updated based on the loss function.

On the basis of the pre-trained feature extraction layer, training the stage identification layer may reduce a data volume of the first training sample and the label of the first training sample.

In some embodiments, the first training sample may include a sample audio recognition result. In some embodiments, the embedding layer may also be trained. For example, the trained embedding layer may be obtained by training a judgment model. The judgment model may include an embedding layer and a judgment layer. The judgment model may be configured to judge a type of the audio, such as a language about thoracotomy and blood transfusion. Similar to the feature extraction layer of the above-mentioned pre-training, the stage identification layer may be trained based on the trained embedding layer. The sample audio recognition result may be input to the trained embedding layer, and then the sample audio feature output from the embedding layer may be input to the stage identification layer to be trained.

In some embodiments, the first training sample may include sample physiological monitoring data, type(s) of sample surgical equipment, usage data of the sample surgical equipment, or the like, or a combination thereof.

In some embodiments, the first training sample(s) and the label(s) of the first training sample(s) may be obtained based on historical data. For example, the first training sample(s) and the label(s) of the first training sample(s) may be obtained based on historical photographed image(s), historical communication audio data, storage data of historical physiological monitoring equipment, parameter(s) of historical surgical equipment, or other data in historical surgical process(es).

In some embodiments, the recognition model may be obtained based on joint training of the feature extraction layer and the stage identification layer. The identification module 220 may train the initial feature extraction layer and the initial stage identification layer based on the first training sample and the label of the first training sample to obtain a trained recognition model.

The first training sample(s) may include sample video data, sample audio recognition result(s), sample physiological monitoring data, type(s) of sample surgical equipment, and usage data of the sample surgical equipment. The label(s) may include an actual surgical stage. Specifically, the sample video data with the label(s) may be input to the feature extraction layer to obtain the sample video image feature output by the feature extraction layer. Then, the sample video image feature, the sample voice feature, the sample physiological monitoring data, the type(s) of the sample surgical equipment and the usage data of the sample surgical equipment output by the feature extraction layer may be input to the stage identification layer to obtain the stage recognition result output by the stage identification layer. The loss function may be constructed based on the output of the stage identification layer and the label, and the parameter(s) of each layer in the recognition model may be updated based on the loss function to obtain the trained recognition model.

In some embodiments, the first training sample(s) may be obtained based on historical data. More descriptions may be found in the acquisition method of the first training sample(s).

As mentioned above, based on different types of the surgical operations, different recognition models may be configured to obtain the different surgical stages corresponding to the different types of the surgical operation. Therefore, the training of different recognition models may need to be implemented based on different training samples. For example, for a thoracotomy, the identification module 220 may train the recognition model based on the first training sample(s) of the thoracotomy and label(s) of the thoracotomy. The first training sample(s) of the thoracotomy may include historical video data in a historical thoracotomy and corresponding historical first auxiliary data. For another example, for a cholecystectomy, the identification module 220 may train the recognition model based on the first training sample(s) of the cholecystectomy and label(s) of the cholecystectomy. The first training sample(s) of the cholecystectomy may include historical video data and corresponding historical first auxiliary data. The training methods for different recognition models corresponding to different types of the surgical operations may be the same. More description of a specific model training method may be found in the above description of model training.

Based on the video data and the first auxiliary data, the surgical stage may be identified by the recognition model, thus in response to the surgical stage, an activation of the surgical equipment used for a surgical operation may be triggered and/or a guidance of the surgical operation may be provided. Thus, the surgical automation may be implemented, the time of manual operation may be saved, and the guidance of the surgical operation may be provided to the doctor in time, thus the success rate of the surgical process may be improved.

It should be noted that the above description of the method for identifying the surgical stage of the surgical process is merely for illustration, and does not limit the scope of the present disclosure. For those skilled in the art, under the guidance of the present disclosure, various modifications and changes may be made to the method for identifying the surgical stage of the surgical process. However, these modifications and changes are still within the scope of the present disclosure.

FIG. 5 is a flowchart illustrating an exemplary process of determining target guidance information according to some embodiments of the present disclosure. In some embodiments, process 500 may be performed by the first determination module 240.

In some embodiments, determining the target guidance information may include determining the target guidance information 520 from a plurality of pieces of standard guidance information based on a matching relationship 510 between the surgical stage and reference information corresponding to the plurality of pieces of standard guidance information. The reference information may include a reference surgical stage.

The standard guidance information may refer to information that may be configured to guide a doctor to perform a surgical operation. In some embodiments, the standard guidance information may include a type of the surgical operation, the surgical equipment and a parameter, the surgical operation, a corresponding site of the surgical operation, patient's case information, physiological data in the surgical process, or the like.

In some embodiments, the standard guidance information may be generated based on a variety of information. In some embodiments, the standard guidance information may be generated based on the guidance of the surgical operation, academic discussions of a plurality of authoritative surgeons, or the like. For example, the standard guidance information may be generated based on the standard surgical operation demonstration of the plurality of authoritative surgeons, notes or summaries, such as words or voice of the plurality of authoritative surgeons.

In some embodiments, the standard guidance information may be generated based on a historical surgical record that meets a predetermined condition.

The historical surgical record may refer to a record of a completed surgery process. The historical surgical record may include a text record, a voice record, a 2D video or a 3D video of the surgical process. In some embodiments, the historical surgical record may be obtained from a record or log of at least one surgical equipment.

In some embodiments, the predetermined condition met by the historical surgical record may include that the historical surgical process is successful, or the historical surgical process is a gold standard of an authoritative surgeon, or the risk of the historical surgical record is less than a predetermined value. In some embodiments, the predetermined condition may be manually set, or may be obtained by a historical predetermined condition met by the historical surgical record.

In some embodiments, the standard guidance information may be generated by identifying the historical surgical record. For example, a plurality of historical surgical stage and data such as doctor's surgical operation, a parameter of a surgical equipment corresponding to each historical surgical stage may be determined by identifying a 2D video or a 3D video image of the historical surgical record. Thus, the standard guidance information may be generated. For example, for an opening chest stage of a thoracotomy, an incision position and a size and a direction of the incision in the historical surgical record may be identified and used as the guidance of the surgical operation in the standard guidance information. Meanwhile, the parameter of the surgical equipment, such as the power of the high-frequency electric knife, may be obtained and used as the parameter of the surgical equipment in the standard guidance information. In some embodiments, the standard guidance information may also be manually generated based on the historical surgical record. For example, a doctor may discuss and summarize the guidance displayed in the historical surgical record by watching the historical surgical record.

The method for obtaining the standard guidance information may be broadened by generating the standard guidance information based on historical surgical record, and more reference guidance information may be provided, thus a doctor may find the most matching standard guidance information to guide a smooth surgical process.

The reference information corresponding to the plurality of pieces of the standard guidance information may refer to specific contents of the standard guidance information that may be used for guiding a surgical operation. In some embodiments, the reference information may include a reference surgical stage.

The reference surgical stage may refer to a surgical stage corresponding to the standard guidance information. For example, when the standard guidance information is a method for cholecystectomy, a corresponding reference surgical stage may be a cutting a gallbladder off stage.

In some embodiments, the reference information may include other information, such as the first reference physiological data and/or first reference case data. More descriptions may be found in operation 620 in FIG. 6.

The matching relationship 510 between the surgical stage and the reference information corresponding to the plurality of pieces of standard guidance information may refer to a similarity level between the surgical stage and the reference information corresponding to the plurality of pieces of standard guidance information, for example, the similarity level between the surgical stage and a reference surgical stage. In some embodiments, the matching relationship may be expressed by a matching degree. For example, the matching degree may be 60%, 80%, or a literal expression of “match” or “mismatch”. In some embodiments, the matching relationship may also be classified based on the matching degree. For example, the matching relationship may be classified from low to high into first-level matching relationship to third-level matching relationship based on the matching relationship. In some embodiments, the matching relationship may be determined by a stage recognition result. If the stage recognition result is consistent with the reference surgical stage, the surgical stage may match the reference surgical stage. If the stage recognition result is not consistent with the reference surgical stage, the surgical stage may not match the reference surgical stage.

In some embodiments, the matching relationship may be determined in combination with the first reference physiological data and/or the first reference case data. For example, the matching degree may be determined based on the similarity level between the physiological data and/or the case data of the patient and the first reference physiological data and/or the first reference case data. In some embodiments, a vector may be constructed based on information such as the surgical stage, physiological data and/or case data, and the matching degree may be determined by calculating a distance between a vector of the reference information and a vector of the surgical stage. For example, the vector \vec{r}=(s, p, q . . . , . . . ) may be constructed based on the surgical stage, physiological data and/or the case data, where s represents the surgical stage, such as the surgical suturing stage, and other elements such as p, q may represent a kind of physiological data or case data. For example, p represents heart beat data, q represents blood pressure data, or the like. The first determination module 240 may determine the matching degree by calculating the distance between the vector of the reference information and the vector of the patient information of the surgical stage. The distance between vectors may include cosine distances, Euclidean distance, Hamming distance, or the like.

The target guidance information 520 may refer to information that may be configured to guide the surgical stage in the plurality of pieces of the standard guidance information. The target guidance information 520 may include various forms such as video, images. More descriptions of target guidance information may be found elsewhere in the present disclosure, for example, FIG. 3 and the descriptions thereof.

In some embodiments, the target guidance information may be determined based on the matching relationship between the surgical stage and the reference information corresponding to the plurality of pieces of standard guidance information. For example, the standard guidance information with the highest matching degree with the surgical stage may be designated as the target guidance information.

In some embodiments, a plurality of target guidance information may be determined based on the matching relationship. For example, all the standard guidance information corresponding to the three-level matching relationship may be designated as the target guidance information. For another example, the standard guidance information corresponding to the reference information in which the matching degree of the surgical stage is greater than a predetermined threshold of the matching degree may be designated as the target guidance information based on the predetermined threshold of the matching degree. For example, the predetermined threshold of the matching degree may be designated as 70%, and the standard guidance information corresponding to the reference information with the matching degree higher than 70% may be designated as the target guidance information.

In some embodiments, the plurality of target guidance information may be displayed to the user through a user terminal. For example, the plurality of target guidance information may be ordered and displayed based on the matching degree, and the target guidance information with the highest matching degree may be displayed at a top level. For another example, the plurality of target guidance information may be displayed with different classifications based on different expression forms of the target guidance information. For example, a display interface of the user terminal may be divided into a plurality of areas, and the plurality of areas may respectively display video information, image information, text information at the same time. When the target guidance information is displayed in a corresponding area, a matching degree of the target guidance information may be displayed at the same time. For the target guidance information with the highest matching degree, a proportion of the corresponding area in the display interface may be the largest. The user may click one of the target guidance information on the user terminal to watch detailed information.

The target guidance information may be determined based on the matching relationship between the surgical stage and the reference information corresponding to the plurality of pieces of the standard guidance information, and the standard guidance information with a high matching degree may be determined as the target guidance information. Thus, the target guidance information may meet the needs of the doctor better, and the surgical process may be guided better and more accurately.

FIG. 6 is another flowchart illustrating an exemplary process of determining target guidance information according to some embodiments of the present disclosure. Process 600 may be performed by the first determination module 240.

In 610, second auxiliary data may be obtained. The second auxiliary data may include case data of a subject, physiological monitoring data related to an acquisition time of the video data, or the like, or a combination thereof.

The second auxiliary data may refer to data configured to assist in determining the target guidance information. In some embodiments, the second auxiliary data may include data related to a surgical object. For example, the second auxiliary data may include case data of the surgical object, physiological monitoring data related to the acquisition time of the video data, or other data.

The surgical object may refer to a patient who is undergoing a surgical process, such as a patient, or an injured.

The case data may refer to a record of disease development of the surgical object and the corresponding examination, diagnosis or treatment. For example, the case data may include a historical diagnosis and treatment record and a medication record of the surgical object.

The acquisition time of video data may refer to a time when video data is obtained using a camera. More descriptions of acquisition time may be found elsewhere in the present disclosure, for example, FIG. 4 and the descriptions thereof.

The physiological monitoring data may refer to physiological data of the surgical object that needs to be monitored during the surgical process. For example, the physiological monitoring data may include data such as respiration, heartbeat, pulse, or blood pressure of the surgical subject. The physiological monitoring data may be different in different surgical operations or for different surgical objects. For example, for a diabetes patient, the physiological monitoring data may also include blood glucose data.

In some embodiments, the physiological monitoring data may be obtained based on the corresponding medical monitoring equipment. For example, the blood pressure data may be obtained based on a sphygmomanometer. In some embodiments, the medical monitoring equipment may store the physiological monitoring data in real time. For instance, the physiological monitoring data may be stored in the storage device of the medical monitoring equipment, or the physiological monitoring data may be uploaded to a remote server for storage. In some embodiments, when obtaining the physiological monitoring data, the medical monitoring equipment may store the information of the surgical object (e.g., an ID number of the surgical object) and the corresponding acquisition time jointly.

The physiological monitoring data related to the acquisition time may refer to the physiological monitoring data of the corresponding surgical object at the time of obtaining the video data. For example, if the acquisition time of a piece of video data is from 9:00 a.m. to 10:00 a.m., the physiological data of the surgical object monitored during the period from 9:00 a.m. to 10:00 a.m. may be the physiological monitoring data related to the acquisition time of the video data.

In some embodiments, the second auxiliary data may be obtained based on a variety of ways.

For example, for the case data of the surgical object, the first determination module may directly retrieve the corresponding case data from a database based on identification data of the surgical object, or generate the corresponding case data based on description(s) of the surgical object.

For another example, for the physiological monitoring data related to the acquisition time of the video data, the first determination module may first obtain the acquisition time of the video data based on the video data, and then retrieve the physiological monitoring data of the surgical object at the corresponding acquisition time from a storage device based on the acquisition time of the video data. For example, if the physiological monitoring data and the video data during the surgical process may be stored jointly based on the acquisition time, the first determination module may obtain the corresponding physiological monitoring data with the video data from the database.

In 620, the target guidance information may be determined based on the surgical stage, and a matching relationship between the second auxiliary data and the reference information.

In some embodiments, the reference information corresponding to the plurality of pieces of standard guidance information may further include first reference physiological data, first reference case data, or the like, or a combination thereof.

The first reference physiological data may refer to the physiological monitoring data of the surgical subject corresponding to the standard guidance information of the surgical process. The first reference physiological data may be physiological monitoring data of the surgical subject before the surgical process, for example, a blood pressure, or a blood glucose of the surgical subject before the surgical process.

The first reference case data may refer to the case data of the surgical object corresponding to the standard guidance information. For example, the first reference case data may be case data of the surgical object corresponding to a standard bypass surgery.

The matching relationship between the second auxiliary data and the reference information may refer to a similarity level between the second auxiliary data and the reference information. In some embodiments, the matching relationship may include a match, a mismatch, or the like. In some embodiments, a similarity threshold may be predetermined. If the similarity level between the second auxiliary data and the reference information exceeds the predetermined similarity threshold, the matching relationship between the second auxiliary data and the reference information may be considered as matching, otherwise, mismatching.

The similarity level between the second auxiliary data and the reference information may be determined in various ways. For example, both the second auxiliary data and the reference information may be represented as a vector, and then a distance between a vector of the second auxiliary data and a vector of the reference information may be determined. The similarity level may be determined based on the distance. The smaller the distance is, the greater the similarity level is. The distance may include a Manhattan distance, a Euclidean distance, a Chebyshev distance, a cosine distance, a Mahalanobis distance, or the like.

The surgical stage may refer to a current surgical stage, which may be determined based on a current surgical progress or time. More descriptions of the surgical stage may be found elsewhere in the present disclosure, for example, FIG. 3 and the descriptions thereof.

In some embodiments, the first determination module may determine the target guidance information based on the matching relationship among the second auxiliary data, the reference information, and the surgical stage. For example, the first determination module may designate the guidance information corresponding to the reference information matched with the second auxiliary data as candidate guidance information. And in combination with the surgical stage, the candidate guidance information corresponding to a same surgical stage may be designated as the target guidance information among the candidate guidance information.

For example, if second auxiliary data A matches the reference information A, the reference information B, and the reference information C, and the surgical stage is a preoperative preparation stage, then a surgical stage corresponding to the reference information B may be the preoperative preparation stage, and guidance information corresponding to the reference information B may be designated as the target guidance information.

In some embodiments of the present disclosure, the target guidance information may be determined based on the case data, the physiological monitoring data and the surgical stage of the surgical object. Thus, the target guidance information may be more consistent with an actual situation, and may be more instructive.

FIG. 7 is a flowchart illustrating an exemplary process of providing prompt information according to some embodiments of the present disclosure. In some embodiments, process 700 may include the following operations:

In 710, execution data may be acquired. The execution data may include at least one of a video image of an execution process of the surgical operation or usage data of the surgical equipment used for the surgical operation generated in the execution process. In some embodiments, operation 710 may be performed by the second acquisition module 250.

The execution data may refer to data acquired during the surgical process. The execution data may be data related to the surgical operation and the surgical equipment, and the execution data may also be data related to the surgical object.

In some embodiments, the execution data may include a video image of an execution process of the surgical operation, usage data of the surgical equipment used for the surgical operation generated in the execution process, or the like, or a combination thereof. The video image of the execution process of the surgical operation may refer to recorded video image data of the surgical process. For example, the video image of the execution process may include an operation video of the doctor during the surgical operation, a monitoring video of the surgical object, or the like. The usage data of the surgical equipment used for the surgical operation generated in the execution process may refer to usage data of the surgical equipment recorded during the surgical operation. For example, the usage data of the surgical equipment may include a time of using the surgical equipment, a parameter of the surgical equipment, or the like.

The execution data may be acquired in a variety of ways. For example, the video image of the execution process of the surgical operation may be acquired based on the video recorded during the surgical operation. For another example, the usage data of the surgical equipment used for the surgical operation generated in the execution process may be acquired based on the recorded usage data of the surgical equipment. For another example, the usage data of the surgical equipment used for the surgical operation generated in the execution process may be acquired based on an image analysis of the video recorded during the surgical operation, or acquired based on usage information input by a user of the surgical equipment.

In 720, an execution level of the surgical operation may be determined based on a matching relationship between the execution data and the target guidance information. In some embodiments, operation 720 may be performed by the second determination module 260.

The matching relationship between the execution data and the target guidance information may refer to a matching level between the execution data and the target guidance information. In some embodiments, different matching relationships may be determined based on different matching levels. In some embodiments, the matching relationship may be represented based on a value, a level, or the like. For example, the matching relationship may be completely matching, relatively matching, mismatching, or the like. For another example, the matching relationship may be expressed based on a value of 0-10. The larger the numerical value is, the higher the matching level is.

In some embodiments, the matching relationship between the execution data and the target guidance information may be obtained based on a similarity level between the execution data and the target guidance information. For example, a judgment model of the similarity level may be configured to determine the similarity level between the execution data and the target guidance information. The judgment model of the similarity level may include a feature extraction layer and/or a similarity judgment layer. The execution data and the target guidance information may be designated as an input of the feature extraction layer to obtain a feature vector of the execution data and a feature vector of the target guidance information, and then the two feature vectors may be input to the similarity judgment layer to obtain the similarity level between the execution data and the target guidance information. The feature extraction layer may be of a CNN structure, or the like. The similarity judgment layer may be of a neural network (NN) structure, or a deep neural network (DNN) structure.

The execution level may refer to an execution level of the target guidance information, and the execution level may be determined based on the matching relationship between the execution data and the target guidance information. In some embodiments, the execution level may be expressed based on various forms. For example, the execution level may be expressed by a percentage in a range of 0-100%. The larger a value of the percentage is, the higher the execution level of the target guidance information is.

In some embodiments, different execution levels corresponding to different matching relationships between the execution data and the target guidance information may be predetermined, and an execution level comparison table may be generated. After determining the matching relationship between the execution data and the target guidance information, the corresponding execution level may be determined based on the execution level comparison table. For example, the execution level may be determined as 100% when the matching relationship is full matching.

In some embodiments, the execution data may further include physiological monitoring data of the execution process, and the reference information may further include second reference physiological data. In some embodiments, the execution level may be determined based on the matching relationship among the execution data, the target guidance information and the second reference physiological data.

The physiological monitoring data in the execution process may refer to data obtained by monitoring a physiological feature of the surgical object during the surgical process. For example, the physiological monitoring data of the execution process may include blood pressure data, respiratory data, or the like, of the surgical object during the surgical process.

The second reference physiological data may refer to the physiological monitoring data during a surgical operation of the surgical object corresponding to the standard guidance information. For example, the second reference physiological data may be the physiological monitoring data, such as the respiration, heartbeat, pulse and blood pressure of the surgical object during a standard bypass operation.

The matching relationship among the execution data, the target guidance information and the second reference physiological data may refer to the matching level between relevant data in the execution data and data corresponding to the target guidance information and the second reference physiological data respectively. For example, the matching relationship among the execution data, the target guidance information and the second reference physiological data may include a matching level between a video image of the execution process and a corresponding video image in the target guidance information, a similarity level between the usage data of the surgical equipment in the execution process and the usage data of the surgical equipment in the target guidance information, and a similarity level between the physiological monitoring data of the execution process and the second reference physiological data.

In some embodiments, similar to an expression of the matching relationship between the execution data and the target guidance information, the matching relationship among the execution data and the target guidance information and the second reference physiological data may also be expressed based on a value, or the like. More descriptions of the matching relationship between the execution data and the target guidance information may be found elsewhere in the present disclosure.

In some embodiments, the execution level may be determined based on different matching relationships among the execution data, the target guidance information, and the second reference physiological data. More descriptions may be found in descriptions of determining the execution level based on the matching relationship between the execution data and the target guidance information.

In some embodiments, a final execution level may be determined based on weighting and summing a plurality of execution levels. More descriptions may be found elsewhere in the present disclosure, for example, FIG. 8 and the descriptions thereof.

In some embodiments of the present disclosure, the execution level may be determined based on the matching relationship among the execution data, the target guidance information, and the second reference physiological data. The execution level of the surgical operation may be determined based on an actual physiological performance of the surgical object in the surgical operation, thereby making the execution level more accurate.

In 730, in response to that the execution level is less than a threshold, prompt information may be provided. In some embodiments, operation 730 may be performed by the prompt module 270.

The threshold may be a value of a minimum execution level that a predetermined execution level needs to meet. For example, the threshold may be a value such as 60%. The value of the threshold may be determined based on an actual situation, and the threshold may be different for different types of the surgical operations.

The prompt information may be information for prompting a doctor when the execution level is lower than the threshold. The prompt information may be in many forms. For example, the prompt information may be audio information, a music melody, or light information, text information, or the like. For example, the prompt information may be an audio prompt that “a current surgical operation is performed at a low level, please pay attention”, or a light that displays with a specific color.

The prompt information may be provided in a variety of ways. For example, the audio information may be played based on equipment worn by the doctor or audio playing equipment installed in the operating room. For example, the light information may be emitted based on a lamp installed in the operating room.

In some embodiments, the process 700 may further include generating new standard guidance information based on the execution data in response to the execution level satisfying a predetermined condition.

The predetermined condition may be that the execution level is higher than a standard value of the execution level. For example, the standard value of the execution level is 90%. If the execution level of a surgical operation is higher than 90%, the execution level of the surgical operation may meet the predetermined condition, and new standard guidance information may be generated based on the execution data of the surgical process. More descriptions of the execution data may be found in the operation 710 in the present disclosure.

Generating new standard guidance information may refer to designating the execution data of the surgical process whose execution level meets the predetermined condition as standard guidance information. For example, for a cholecystectomy, if the execution level of the surgical operation of the cholecystectomy is higher than 90% (i.e., the standard value of the execution level), the execution data obtained based on the surgical operation of the cholecystectomy may be designated as the new standard guidance information of the cholecystectomy.

In some embodiments of the present disclosure, in combination with the execution level of a surgical operation, new standard guidance information may be generated based on the surgical operation with a high execution level, so that the standard guidance information may be continuously updated, and then standard guidance information meeting more scenes may be generated.

In some embodiments of the present disclosure, the execution level of the target guidance information may be determined in combination with the execution data, to judge whether the surgical operation of the doctor and the usage of the surgical equipment are abnormal. Then, prompt information may be provided in time when the surgical process deviates from the target guidance information, facilitate the doctor to correct an error and improve the success rate of the surgical operation.

In some embodiments, the second determination module 260 may determine the execution level of the surgical operation based on a variety of ways. For example, the second determination module 260 may determine the execution level of the surgical operation based on a first execution level, a second execution level and a third execution level, and may also determine the execution level of the surgical operation based on weighting and summing the first execution level, the second execution level and the third execution level.

FIG. 8 is a schematic diagram illustrating an exemplary process of determining an execution level by weighting according to some embodiments of the present disclosure. In some embodiments, operations in the FIG. 8 may be performed by a second determination module 260.

A first execution level 810 may refer to an execution level determined based on a similarity level between the video image feature 401 and the video image feature in the target guidance information. In some embodiments, the first execution level may be expressed in variety of forms. For example, the first execution level may be expressed by a percentage in 0-100%. The larger a value of the percentage is, the higher the first execution level is. For another example, the execution levels may be classified into levels 1-3 based on values of the execution levels from low to high.

The video image feature 401 may be the video image feature output by the feature extraction layer of the recognition model. More descriptions of video image feature may be found elsewhere in the present disclosure, for example, FIG. 4 and the descriptions thereof. The similarity level between the video image feature 401 and the video image feature in the target guidance information may be expressed by a percentage in 0-100%. The larger a value of the percentage is, the higher the similarity level between the video image feature 401 and the video image feature in the target guidance information is. In some embodiments, feature vectors may be constructed to measure the similarity level based on a distance the feature vectors. The shorter the distance is, the higher the similarity level is. More descriptions of constructing the feature vectors may be found elsewhere in the present disclosure, for example, FIG. 4 and the descriptions of the recognition model.

In some embodiments, the second determination module 260 may determine the first execution level in a variety of ways. For example, the first execution level may be determined based on the similarity level between the video image feature 401 and the video image feature in the target guidance information. For example, an execution level comparison table corresponding to a plurality of similarity levels between the video image features 401 and the video image features in the target guidance information may be predetermined. For example, the execution level may be 70% when a predetermined similarity level is 70%.

The second execution level 820 may refer to an execution level determined based on the similarity level between the usage data of the surgical equipment used for the surgical operation generated in the execution process and a parameter of the surgical equipment used for the surgical operation in the target guidance information. In some embodiments, the second execution level may be expressed in a variety of forms. More descriptions of the expression form of the second execution level may be found in the relevant descriptions of the first execution level.

In some embodiments, the usage data of the surgical equipment used for the surgical operation generated in the execution process and the parameter of the surgical equipment used for the surgical operation in the target guidance information may include data such as an arrangement orientation, a working parameter, and a use time of the surgical equipment. More descriptions may be found elsewhere in the present disclosure, for example, FIG. 3, FIG. 7, and relevant descriptions of the surgical equipment used for the surgical operation.

In some embodiments, the similarity level between the usage data of the surgical equipment used for the surgical operation generated in the execution process and the parameter of the surgical equipment used for the surgical operation in the target guidance information may be expressed by a value. The greater the value is, the higher the similarity level is. In some embodiments, a parameter vector may be constructed to measure the similarity level. For example, a vector \vec{n}=(d, e, f) may be constructed based on the usage data of the surgical equipment for surgical operation, where d represents an arrangement orientation of the surgical equipment, e represents a use time of the surgical equipment, and f represents a working parameter. The second determination module 260 may calculate a distance between two parameter vectors corresponding to the working parameter of the surgical equipment used for the surgical operation in the target guidance information and the usage data of the surgical equipment used for the surgical operation generated in the execution process, and measure the similarity level based on the distance. The shorter the distance is, the higher the similarity level is. The distance may include a cosine distance, a Euclidean distance, a Hamming distance, or the like.

In some embodiments, the second determination module 260 may determine the second execution level in a variety of ways. For example, the second execution level may be determined based on the similarity level between the usage data of the surgical equipment used for the surgical operation generated in the execution process and the parameter of the surgical equipment used for the surgical operation in the target guidance information. For example, the execution level comparison table corresponding to the similarity level may be predetermined. After the plurality of similarity levels are determined, the execution level may be determined based on the execution level comparison table of the plurality of similarity levels.

The third execution level 830 may refer to an execution level determined based on the similarity level between physiological monitoring data generated in the execution process and the second reference physiological data. The physiological monitoring data generated in execution process may refer to physiological data of the surgical object that needs to be monitored during execution process. More descriptions of the physiological monitoring data generated in the execution process may be elsewhere in the present disclosure, for example, FIG. 6 and the descriptions thereof. The second reference physiological data may refer to the physiological monitoring data during a surgical operation of the surgical object corresponding to the standard guidance information. More descriptions of the second reference physiological data may be found elsewhere in the present disclosure, for example, FIG. 7 and the descriptions thereof. In some embodiments, the third execution level may be expressed in a variety of forms. More descriptions of the expression form of the third execution level may be found in related descriptions of the first execution level.

In some embodiments, the similarity level between the physiological monitoring data generated in the execution process and the second reference physiological data may be expressed by a value. In some embodiments, the second determination module 260 may determine the third execution level based on a variety of ways. For example, the third execution level may be determined based on the similarity level between the physiological monitoring data generated in execution process and the second reference physiological data.

Merely by way of example, an execution level comparison table corresponding to the plurality of similarity levels between the physiological monitoring data of the execution process and the second reference physiological data may be predetermined, and the execution level may be determined based on the execution level comparison table. The similarity level may be determined by constructing a parameter vector. For example, a vector \vec{l}=(i,j,k, . . . ) may be constructed based on the physiological monitoring data, where each vector element represents a kind of physiological monitoring data, such as respiration, heartbeat, pulse, blood pressure, or the like. The second determination module 260 may calculate a distance between two parameter vectors corresponding to the physiological monitoring data generated in the execution process and the second reference physiological data, and measure the similarity based on a distance between the two parameter vectors. The shorter the distance is, the higher the similarity level is.

In some embodiments, the second determination module 260 may determine a final execution level 840 based on weighting and summing the plurality of execution levels.

The final execution level 840 may refer to an execution level determined based on the first execution level, the second execution level and the third execution level. In some embodiments, the final execution level may be expressed in various forms. For example, the final execution level may be expressed by a percentage in 0-100%. The larger a value of the percentage is, the higher the execution level of the target guidance information is. For another example, the final execution levels may be classified into levels 1-3 based on values of the final execution levels from low to high.

In some embodiments, the second determination module 260 may determine the final execution level by weighting and summing. A formula of weighting and summing may be expressed as P=A×E+B×F+C×G, where P represents the final execution level. A, B and C represent the first execution level, the second execution level and the third execution level respectively. E, F and G represent weights corresponding to the first execution level, the second execution level and the third execution level respectively. For example, the first execution level may be 70%, and a weight of the first execution level may be 0.4. The second execution level may be 60%, and a weight of the second execution level may be 0.3. The third execution level may be 80%, and a weight of the third execution level may be 0.3. Thus, the final execution level obtained by weighting and summing may be 70%.

In some embodiments, a weight of an execution level may be determined based on a variety of ways. For example, the execution level may be predetermined manually. In some embodiments, the weight of the execution level may also be determined based on the type of the surgical operation. For example, a weight comparison table of execution levels corresponding to different types of different surgical operations may be predetermined. For another example, a predetermined weight may also be changed based on a complexity of different types of different surgical operations. For example, based on the predetermined weight, the weight of the first execution level of a surgical operation with a high complexity may be increased, and the weight of the corresponding second execution level and the third execution level may be reduced. The weight of the second execution level of a surgical operation with a high equipment accuracy may also be increased, and the weight of the corresponding first execution level and the third execution level may be reduced.

The final execution level may be determined based on the first execution level, the second execution level and the third execution level, and the final execution level may be determined based on data such as video image features, usage data of the surgical equipment used for the surgical operation, physiological monitoring data, or the like. The final execution level may be determined by fully considering various factors affecting the final execution level, thus the determination of the final execution level may be more reasonable and more accurate, to better judge whether there is a problem in the surgical process.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Meanwhile, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution—e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the present disclosure object requires more features than the features mentioned in the claims. Rather, claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Contents of each of patents, patent applications, publications of patent applications, and other materials, such as articles, books, specifications, publications, documents, etc., referenced herein are hereby incorporated by reference, excepting any prosecution file history that is inconsistent with or in conflict with the present document, or any file (now or later associated with the present disclosure) that may have a limiting effect to the broadest scope of the claims. It should be noted that if the description, definition, and/or terms used in the appended materials of the present disclosure is inconsistent or conflicts with the content described in the present disclosure, the use of the description, definition and/or terms of the present disclosure shall prevail.

Finally, it should be understood that the embodiments described in the present disclosure merely illustrates the principles of the embodiments of the present disclosure. Other modifications may be within the scope of the present disclosure. Accordingly, by way of example, and not limitation, alternative configurations of embodiments of the present disclosure may be considered to be consistent with the teachings of the present disclosure. Accordingly, the embodiments of the present disclosure are not limited to the embodiments explicitly introduced and described by the present disclosure.

What is claimed is:

1. A method for surgical automation, comprising:
obtaining video data generated in a surgical process using a camera;
identifying a surgical stage in the surgical process based on the video data; and
triggering an activation of a surgical equipment used for a surgical operation and/or providing a guidance of the surgical operation based on the surgical stage.

2. The method of claim 1, wherein identifying a surgical stage in the surgical process includes:
inputting the video data into a recognition model; and
determining the surgical stage based on an output of the recognition model, wherein the recognition model is a machine learning model.

3. The method of claim 2, wherein an input of the recognition model further includes first auxiliary data related to an acquisition time of the video data, the first auxiliary data including at least one of audio data, physiological monitoring data, and a type of a surgical equipment and usage data of the surgical equipment.

4. The method of claim 1, wherein the surgical equipment used for the surgical operation and the guidance of the surgical operation are determined based on target guidance information, and the target guidance information is determined based on the surgical stage.

5. The method of claim 4, wherein determining the target guidance information includes:
determining the target guidance information from a plurality of pieces of standard guidance information based on a matching relationship between the surgical stage and reference information corresponding to the plurality of pieces of standard guidance information, wherein the reference information includes a reference surgical stage.

6. The method of claim 4, further including:
acquiring execution data, wherein the execution data includes at least one of a video image of an execution process of the surgical operation or usage data of the surgical equipment used for the surgical operation generated in the execution process;
determining an execution level of the surgical operation based on a matching relationship between the execution data and the target guidance information; and
in response to that the execution level is less than a threshold, providing prompt information.

7. The method of claim 5, wherein the reference information further includes at least one of first reference physiological data or first reference case data, and determining target guidance information includes:
obtaining second auxiliary data, the second auxiliary data including at least one of case data of a subject or physiological monitoring data related to an acquisition time of the video data; and
determining the target guidance information based on the surgical stage, and a matching relationship between the second auxiliary data and the reference information.

8. The method of claim 5, wherein the plurality of pieces of standard guidance information is generated based on historical surgical record, and the historical surgical record meets a predetermined condition.

9. The method of claim 1, wherein the surgical stage includes at least one of a normal surgical stage or an abnormal surgical accident.

10. The method of claim 1, wherein triggering an activation of a surgical equipment used for a surgical operation includes:
transmitting an activation instruction to the surgical equipment used for the surgical operation, wherein the activation instruction includes one or more parameters.

11. A system for surgical automation, comprising:
a first acquisition module configured to obtain video data generated in a surgical process using a camera;
an identification module configured to identify a surgical stage in the surgical process based on the video data; and
a triggering and displaying module configured to trigger an activation of a surgical equipment used for a surgical operation and/or provide a guidance of the surgical operation based on the surgical stage.

12. The system of claim 11, wherein the identification module is further configured to:
input the video data into a recognition model; and
determine the surgical stage based on an output of the recognition model, wherein the recognition model is a machine learning model.

13. The system of claim 12, wherein an input of the recognition model further includes first auxiliary data related to an acquisition time of the video data, the first auxiliary data including at least one of audio data, physiological monitoring data, and a type of a surgical equipment and usage data of the surgical equipment.

14. The system of claim 11, wherein the system further includes a first determination module, wherein
the first determination module is configured to determine target guidance information based on the surgical stage; and
the triggering and displaying module is further configured to determine the surgical equipment used for the surgical operation and the guidance of the surgical operation based on the target guidance information.

15. The system of claim 14, wherein the first determination module is further configured to:
determine the target guidance information from a plurality of pieces of standard guidance information based on a matching relationship between the surgical stage and reference information corresponding to the plurality of pieces of standard guidance information, wherein the reference information includes a reference surgical stage.

16. The system of claim 14, further including:
a second acquisition module configured to acquire execution data, wherein the execution data includes at least one of a video image of an execution process of the surgical operation or usage data of the surgical equipment used for the surgical operation generated in the execution process;
a second determination module configured to determine an execution level of the surgical operation based on a matching relationship between the execution data and the target guidance information; and
a prompt module configured to, in response to that the execution level is less than a threshold, provide prompt information.

17. The system of claim 15, wherein the reference information further includes at least one of first reference physiological data or first reference case data, and the first determination module is further configured to:
obtain second auxiliary data, the second auxiliary data including at least one of case data of a subject or physiological monitoring data related to an acquisition time of the video data; and
determine the target guidance information based on the surgical stage, and a matching relationship between the second auxiliary data and the reference information.

18. The system of claim 11, wherein the surgical stage includes at least one of a normal surgical stage or an abnormal surgical accident.

19. The system of claim 11, wherein the triggering and displaying module is further configured to:
transmit an activation instruction to the surgical equipment used for the surgical operation, wherein the activation instruction includes one or more parameters.

20. A non-transitory computer readable medium storing at least one set of instructions, wherein when executed by at least one processor of a computing device, the at least one set of instructions directs the at least one processor to:
obtain video data generated in a surgical process using a camera;
identify a surgical stage in the surgical process based on the video data; and
trigger an activation of a surgical equipment used for a surgical operation and/or provide a guidance of the surgical operation.
